(19) United States
(12) Patent Application Publication
(10) Pub. No.: US 2002/0103540 A1
(43) Pub. Date: Aug. 1, 2002

(54) SPINAL FUSION IMPLANT

(76) Inventors: DEREK REDVERS COOPER, BERKSHIRE (GB); RAYMOND CLAIRENCE CHANCE WADEY, WILTSHIRE (GB)

Correspondence Address: YOUNG & THOMPSON, 745 SOUTH 23RD STREET 2ND FLOOR, ARLINGTON, VA, 22202

(21) Appl. No.: 09/460,374

(22) Filed: Dec. 13, 1999

(30) Foreign Application Priority Data
Jan. 16, 1999 (GB) 9900882.3

(51) Int. Cl. A61F 2/44

(52) U.S. Cl. 623/17.16; 623/17.11

(57) ABSTRACT

A spinal fusion implant comprises a rigid hollow elongate member for insertion in a direction parallel to the longitudinal extent of the member between the vertebrae of a human spine, the rigid member having a height (when considered in an use implanted position with a patient's spine vertical) greater than its width w so that the member can be inserted between the two vertebrae and then displaced angularly through 90° to an in use position. The member has a body portion which has a plurality of apertures for allowing bone growth inducing material introduced into the hollow member to extend therethrough and a plurality of arcuate teeth which bite into the vertebrae when the member is in an in use position.

### INTRODUCTION

This invention relates to a spinal fusion implant for insertion between two adjacent vertebrae of a human spine after removal of a damaged spinal disc.

Known spinal fusion implants are generally cylindrical and have to be screwed into place between the end plates of two adjacent vertebrae. This has the disadvantage that cortical bone has to be removed from the vertebrae in order to accommodate the implant. This could expose bone less capable of bearing load. Also the implants can act as rollers which would not provide a stable environment for bony fusion.

### SUMMARY OF THE INVENTION

According to the present invention, there is provided a spinal fusion implant comprising a rigid hollow elongate member for insertion in a direction parallel to the longitudinal extent of the member between the vertebrae of a human spine, the rigid member having a height (when considered in an in use implanted position with the patient's spine vertical) greater than its width so that the member can be inserted between two vertebrae and then displaced angularly through 90° to an in use position, the member having a body portion which has a plurality of apertures for allowing bone growth inducing material introduced into the hollow member to extend therethrough and a plurality of arcuate teeth which bite into the vertebrae when the member is in an in use position.

Preferred and/or optional features of the invention are set forth in claims 2 to 8, inclusive.

The invention will now be more particularly described with reference to the accompanying drawings.

### BRIEF DESCRIPTION OF THE DRAWINGS

FIG. 1 is a plan view of one embodiment of a spinal fusion implant according to the present invention,

FIG. 2 is a section taken along the line A-A of FIG. 1,

FIG. 3 is an end view taken in the direction of arrow B of FIG. 2,

FIG. 4 is a plan view of a tamping jig, and

FIG. 5 is a view in the direction of arrow A of FIG. 4.

### DESCRIPTION OF THE PREFERRED EMBODIMENT

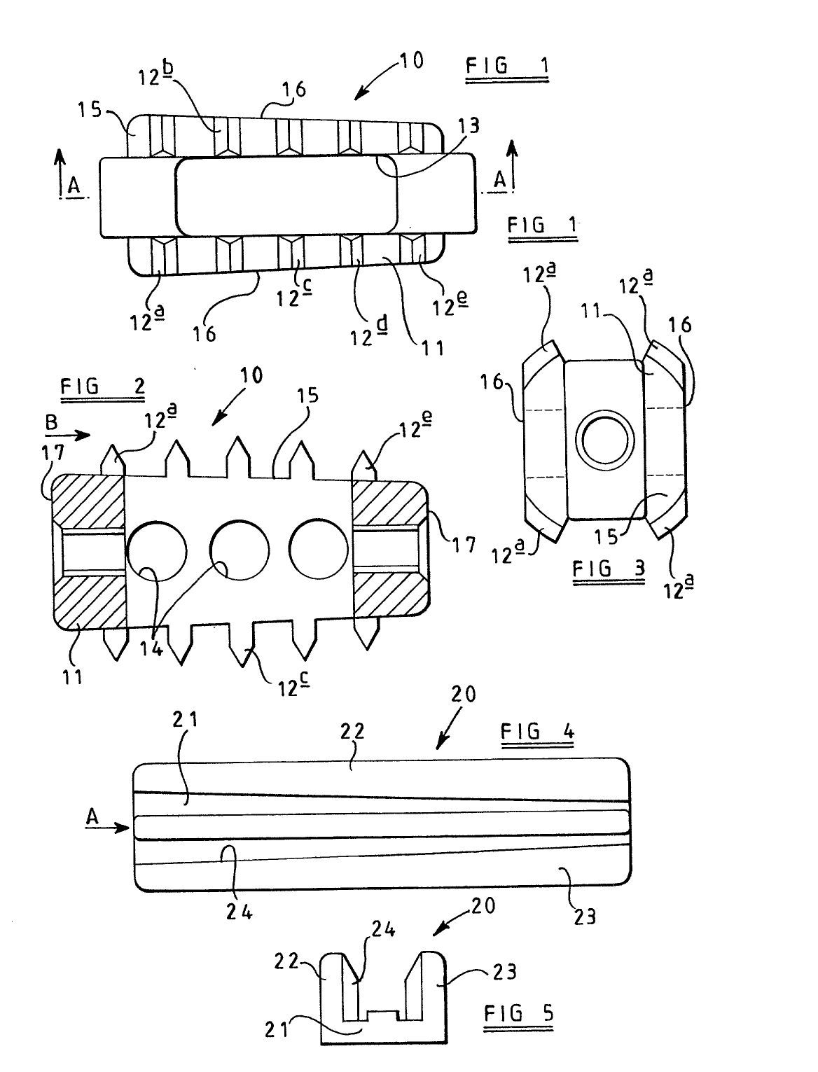

Referring now to FIGS. 1 to 3 of the drawings, the spinal fusion implant shown therein is intended for insertion between the end plates of two adjacent vertebrae of a human spine after removal of damaged spinal disc. The implant comprises a rigid hollow elongate member 10 which has a height h (when considered in an in use implanted position with the patient's spine vertical) greater than its width w so that the member can be inserted between two vertebrae and then displaced angularly through 90° to an in use position. This allows for the smooth entry of the implant into the intervertebral space before angularly displacing the implant to wedge it in place.

The member 10 comprises a body portion 11, which tapers from one end to the other when viewed from the top and the bottom 15 (as shown in FIG. 1) and when viewed from its two sides 16 (as shown in FIG. 2), and a plurality of arcuate teeth 12.

The top and the bottom of the body 11 are each provided with an elongate generally rectangular aperture 13 and each side of the body 11 is provided with three circular apertures 14. These apertures 13 and 14 allow bone growth inducing material introduced into the hollow member 10 to extend therethrough.

There are several sets of teeth 12. In the embodiment shown, there are five sets of teeth 12a to 12e. The sets 12a to 12e are spaced apart in a direction parallel to the longitudinal extent of the rigid member 10. Each set comprises four teeth. Two of the teeth extend from the top of the body portion 11 and are equi-angularly spaced from, and on opposite sides, of a plane p bisecting the width of the rigid member and the other two teeth extend from the bottom of the body portion 11 and are also equi-angularly spaced from, and on opposite sides of, the plane p. As clearly shown in FIGS. 1 and 3, the teeth do not extend beyond the two sides 16 of the body portion 11. This further promotes smooth entry of the implant into the intervertebral space.

As shown clearly in FIG. 3, the sharp edges of the teeth 12 of each set of teeth 12a to 12e lie on a common circle centred about a central longitudinal axis of the implant.

The height of the teeth 12 varies along the longitudinal extent of the implant. The teeth 12c midway or substantially midway between the ends 17 of the rigid member are higher than the teeth 12a and 12e at or adjacent to opposite ends 17 of the implant. This ensures anatomical contact between the implant 10 and the concave surfaces of the end plates of the vertebrae. Moreover, the tapered nature of the implant is designed to restore natural lordosis of the lumbar spine.

The body portion 11 of the implant has threaded apertures 18 at opposite ends, respectively, for attachment of the rigid member 10 to an insertion tool (not shown). This allows the implant to be inserted posteriorly or anteriorly.

The large central cavity in the body portion 11 provides good area surface contact between bone growth inducing material which is compacted into the body portion 11 prior to insertion into the intervertebral space.

FIGS. 4 and 5 show an elongate tamping jig 20 for supporting the member 10 while bone growth inducing material is compacted therein. The jig 20 has a base 21 and two side walls 22 and 23 which define therebetween a tapered passage 24 for receiving and supporting the member 10 with the top or bottom uppermost. This exposes one of the generally rectangular apertures 13 whilst the other aperture 13 is covered by an elongate projection upstanding from the base 21 mid-way between the diverging sides of the passage 24 and whilst the circular apertures 14 are covered by the side walls 22 and 23.

Bone growth inducing material is compacted into the hollow member 10 through the said one generally rectangular aperture 13 whilst the member 10 is supported in the jig 20. The elongate nature of the jig 20 allows members 20 of various different sizes to be supported.

The implant is made of any bio-compatible material which is stronger than bone and is typically made from titanium alloy or non-metallic materials such as carbon fibre composites.

What is claimed is:

1. A spinal fusion implant comprising a rigid hollow elongate member for insertion in a direction parallel to the longitudinal extent of the member between the vertebrae of a human spine, the rigid member having a height (when considered in an in use implanted position with the patient's spine vertical) greater than its width so that the member can be inserted between two vertebrae and then displaced angularly through 90° to an in use position, the member having a body portion which has a plurality of apertures for allowing bone growth inducing material introduced into the hollow member to extend therethrough and a plurality of arcuate teeth which bite into the vertebrae when the member is in an in use position.

2. A spinal fusion implant as claimed in claim 1, wherein the body portion of the rigid member tapers between opposite ends thereof.

3. A spinal fusion implant as claimed in claim 1, wherein the teeth project beyond the top and bottom of the body portion but do not project beyond the two sides of the body portion.

4. A spinal fusion implant as claimed in any claim 1, wherein there are several sets of teeth, the sets being spaced apart in a direction parallel to the longitudinal extent of the rigid member and each set comprising four teeth, a first two teeth being for biting into one vertebra and a second two teeth being for in use biting into the other vertebra.

5. A spinal fusion implant as claimed in claim 4, wherein the first two teeth of each set and the second two teeth of each set are equi-angularly spaced from, and on opposite sides of, a plane bisecting the width of the rigid member.

6. A spinal fusion implant as claimed in claim 1, wherein the height of the teeth varies along the longitudinal extent of the rigid member.

7. A spinal fusion implant as claimed in claim 6, wherein the teeth midway or substantially midway between the ends of the rigid member are higher than the teeth at or adjacent to opposite ends of the rigid member.

8. A spinal fusion implant as claimed in claim 1, wherein the body portion of the rigid member has a threaded aperture for attachment of the rigid member to an insertion tool.

9. A spinal fusion implant as claimed in claim 1, in combination with a jig for supporting the hollow rigid member whilst bone growth inducing material is compacted therein, the jig comprising a base and two side walls which define therebetween a tapered passage.

10. The combination of claim 9, wherein the jig has a projection upstanding from the base midway between the diverging sides of the tapered passage
